(19) United States
(12) Patent Application Publication
(10) Pub. No.: US 2009/0036821 A1
(43) Pub. Date: Feb. 5, 2009

(54) Iontophoretic Transdermal Delivery of Nicotine Salts

(75) Inventor: Pamela M. Lai, Parsippany, NJ (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION

(21) Appl. No.: 12/096,612

(22) PCT Filed: Dec. 18, 2006
(86) PCT No.: PCT/US06/62209

§ 371 Date: Jun. 9, 2008

Related U.S. Application Data
(60) Provisional application No. 60/752,731, filed on Dec. 21, 2005.

(51) Int. Cl. A61N 1/30; A61K 31/465

(52) U.S. Cl. 604/20; 514/343

(57) ABSTRACT

The present invention relates to iontophoretic transdermal delivery of nicotine salts useful for nicotine replacement therapy for an individual in need thereof. The present invention further relates to the iontophoretic transdermal delivery of nicotine maleate and nicotine citrate. Methods of reducing skin irritation generally caused by transdermal nicotine delivery by iontophoretic transdermal delivery of nicotine salts are also disclosed.

### FIELD OF THE INVENTION

This invention relates to the iontophoretic transdermal delivery of nicotine salts. More particularly, this invention relates to the iontophoretic transdermal delivery of nicotine maleate and nicotine citrate useful for nicotine replacement therapy for individuals in need thereof.

### BACKGROUND

It is generally known that active, as well as passive, smoking of tobacco products, such as cigars, cigarettes, pipe tobacco presents serious health risks to the user and those subjected to secondary smoke. It is also know that use of other forms of tobacco, such as chewing tobacco, present serious health risks to the user. In fact, it has been stated that cigarettes alone kill more than 400,000 Americans each year and that smoking is responsible for 30% of all cancer deaths in the United States. Another 50,000 Americans die due to tobacco exposure-related diseases (i.e., lung cancer, cardiovascular disease) resulting from second-hand smoke (persons exposed to environmental tobacco smoke). Tobacco use is the number one cause of death and preventable diseases in the United States. Furthermore, the use of tobacco products in many public environments is becoming increasingly restricted or outright banned.

It is recognized that reducing or quitting tobacco usage is often very difficult for persons accustomed to using tobacco. The difficulty arises in large art from the addictive nature of nicotine. Efforts have therefore been made to provide nicotine substitutes suitable for satisfying a tobacco user's craving, but which avoid the health risks associated with tobacco use. Administration of nicotine to addicted smokers can result in a significant reduction in craving for cigarettes. For instance, transdermal nicotine provides smokers with nicotine, the other 4000 harmful chemicals associated with cigarette smoke are not present. Nicotine patches have been commercially available for several years and have been shown to be effective as an aid to smoking cessation. U.S. Pat. Nos. 5,364,630, and 6,165,497 are exemplary. Daily dosage (5 to 22 mg) is regulated and tapered by using patches of different sizes (3.5 to 30 cm²).

Existing nicotine patches are generally geared to deliver nicotine to an individual in a 24 hour period in an amount that is approximately equivalent to that absorbed by smoking a certain number of cigarettes per day, for instance “one pack per day”, which is equivalent to 20 cigarettes per day. Nicotine delivered via a transdermal patch, however, differs from that delivered via smoking or oral nicotine dosage forms in that there may lagtime in achieving the desired level of nicotine and, once achieve, the nicotine blood levels are maintained at some steady state level. Alternatively, smoking provides very rapid uptake of nicotine and fast clearance from the blood. Thus, transdermal delivery systems could benefit from a reduction in lagtime and a more “pulsatile” delivery mechanism.

The relatively large patch size, however, may cause concern to some consumers as it may be difficult to hide from view, thereby drawing unwanted attention. Alternatively, some consumers may find the patch uncomfortable due to the large surface area of skin being exposed to nicotine which can potentially cause irritation. It is known that delivery of certain compounds across the skin can be enhanced when delivered under the force of a small electrical current, i.e. iontophoresis.

Devices useful for iontophoretic delivery of compounds across the skin are known. Some examples include the devices discussed in U.S. Pat. Nos. 5,571,149; 6,553,255; 6,377,847; and 6,546,283; as well as, EP 0705619A1. There is some suggestion that such devices may be useful for delivering base nicotine transdermally to an individual.

However, in addition to the nicotine base form, nicotine is available in various salt forms, such as hydrochloride, bitartrate and the like. These salt forms may enhance delivery through the skin, and there is some suggestion that they may be particularly useful when used in combination with iontophoresis. Such methods of enhancing nicotine delivery could provide flexibility in patch design as it may allow for modifications to patch size or changes to the amount of nicotine active contained within the patch. Such improvements in patch design could result in a number of benefits to the end user. For instance, a smaller iontophoretic patch or an iontophoretic patch comprising less nicotine active, may provide additional benefits, such as possibly resulting in less irritation to the user and, ultimately, improved compliance with the patch form of NRT. Thus, it is desirable to continue to improve transdermal patch designs to enhance the speed and extent of nicotine delivery to a user in need thereof.

To that end, it has been discovered that certain nicotine salts achieve unexpectedly higher levels of nicotine flux than other salts and may be more useful in readily achieving the desired modifications discussed above.

### SUMMARY OF THE INVENTION

The present invention relates to rapid nicotine delivery to an individual in need thereof wherein the nicotine is in the form of a nicotine salt that is iontophoretically delivered via a transdermal patch. In one embodiment the nicotine salt is selected from the group consisting of nicotine citrate and nicotine maleate or a combination thereof. A method of providing fast nicotine craving relief to an individual in need thereof is also disclosed.

### BRIEF DESCRIPTION OF THE DRAWINGS

FIG. 1 is a graphic representation of the cumulative nicotine delivered over time through transdermal delivery of nicotine base in citrate buffer (pH 5.5) by passive diffusion and by iontophoresis. The dotted line indicates termination of current.

FIG. 2 is a graphic representation of the cumulative nicotine delivered over time through transdermal delivery of nicotine base in HEPES ([4-(2-Hydroxyethyl)-1-piperazine ethanesulfonic acid) buffer (pH 8) by passive diffusion and iontophoresis. The dotted line indicates termination of current.

FIG. 3 is a graphic representation of the cumulative nicotine delivered over time through transdermal delivery of nicotine base and nicotine salts (bitartrate and hemisulfate) by passive diffusion and by iontophoresis.

FIG. 4 is a graphic representation of the cumulative nicotine delivered over time through transdermal delivery of nicotine bitartrate salt in 60 mM HEPES donor buffer and 500 mM citrate donor buffer. The dotted line indicates termination of current.

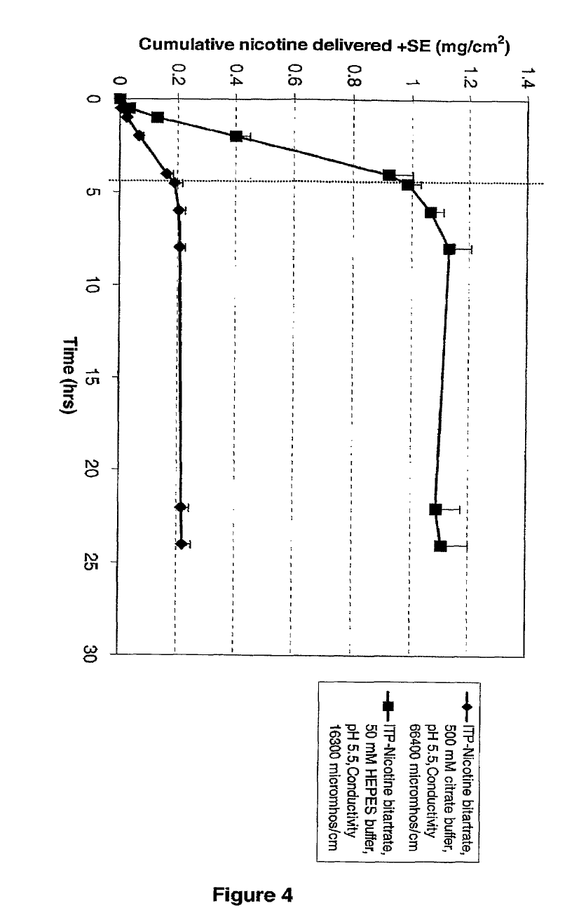

FIG. 5 is a graphic representation of cumulative nicotine delivered over time through iontophoretic delivery of nicotinium dihydro chloride salt and nicotine bitartrate salt. The dotted line indicates termination of current.

FIG. 6 is a graphic comparison of nicotine flux of nicotine bitartrate and nicotinium dihydro chloride salt over time. The dotted line indicates termination of current.

FIG. 7 is a graphic representation of cumulative nicotine delivered over time of iontophoresis of various nicotine salts from 50 mM HEPES buffer, pH 5.5. The dotted line indicates termination of current.

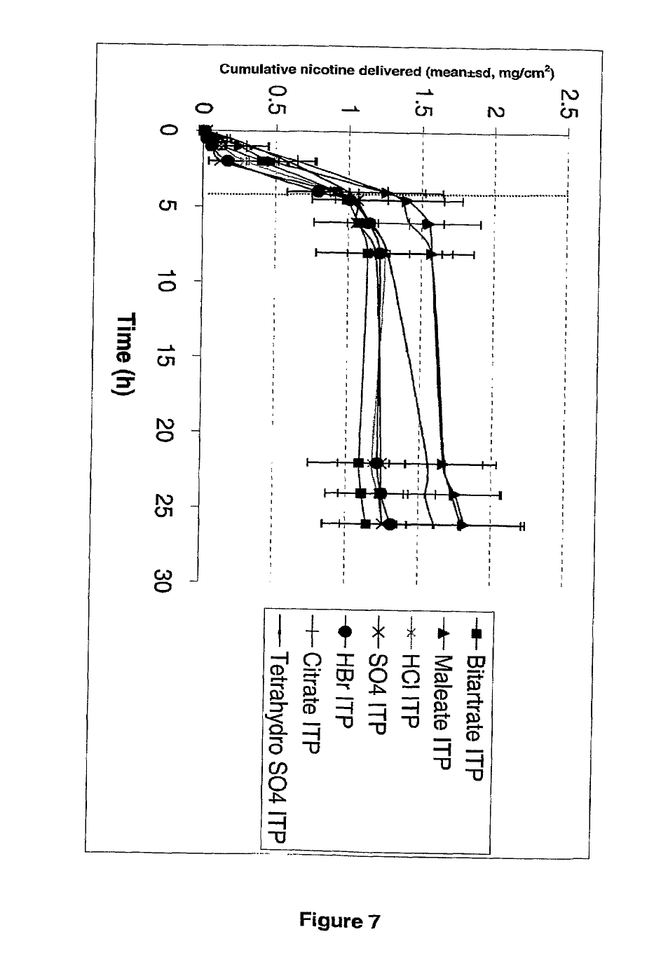

FIG. 8 is a graphic representation of cumulative nicotine delivered over time of passive permeation of nicotine base (pH 8) and iontophoresis of nicotine bitartrate and maleate salts (pH 5.5). The dotted line indicates termination of current.

### DETAILED DESCRIPTION OF THE INVENTION

The present invention relates to iontophoretically enhanced transdermal delivery of nicotine salts. More particularly, this invention relates to the iontophoretic transdermal delivery of certain nicotine salts which have been discovered to have an improved nicotine flux when compared to other nicotine salts and reduced lagtime in delivering nicotine. In particular, nicotine maleate, nicotine citrate and combinations thereof are useful for iontophoretic transdermal delivery of nicotine in a nicotine replacement therapy regimen.

### EXAMPLES

The following methodology was employed in the examples described herein: Dermatomed human skin stored at −80° C. was thawed just prior to use. Each permeation experiment consisted of four replicates. The skin for each replicate was obtained from a different donor so that the variation was randomized. Skin was mounted on Valia-Chien (horizontal) diffusion cells for these in vitro permeation studies, with stratum corneum side facing the donor side. The receptor compartment was filled with pH 7.4 phosphate buffer (50 mM). To the donor compartment, 1% nicotine or its salt was added according to the examples that follow. Prior to use, both donor and receptor solutions were degassed by helium sparging. The temperature of the water bath was set to 32° C. Both donor and receptor compartments were continuously stirred. For iontophoresis, silver wire was used as the anode in the donor compartment and silver/silver chloride as cathode in the receptor compartment. A current of 0.5 mA/sq.cm was applied for 4 hours. Samples taken periodically from the receptor compartment were analyzed by HPLC assay. An Xterra RP18 column was used and the detection wavelength was 261 nm. The mobile phase was 85:15 buffer:acetonitrile, pumped at 1 mL/min and retention time was about 3 minutes.

### Example 1

### Transdermal Delivery of Nicotine Base by Passive Diffusion and Iontophoresis

Permeation of nicotine base was studied under two different sets of conditions.
i) Nicotine in 50 mM HEPES ([4-(2-Hydroxyethyl)-1-piperazine ethanesulfonic acid) buffer pH 5.5 with 50 mM NaCl.
ii) Nicotine in 500 mM Citrate buffer pH 8 with 50 mM NaCl.

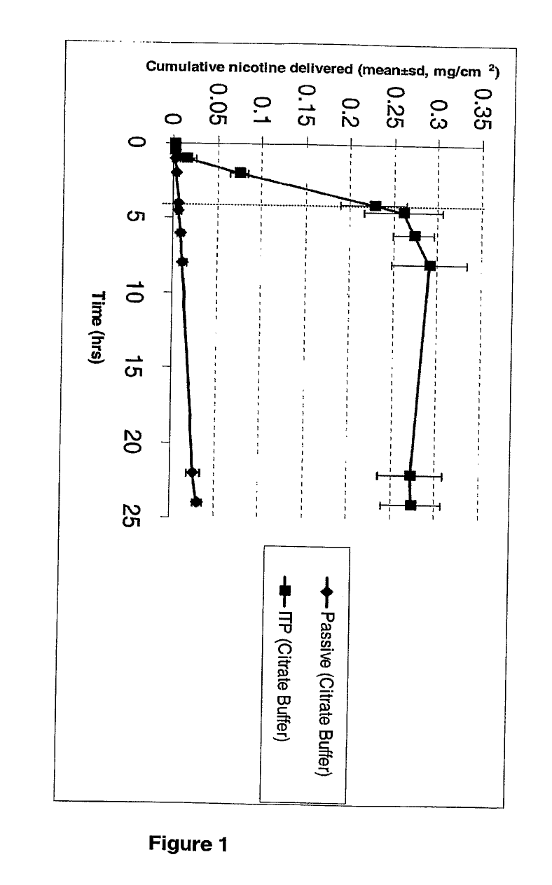

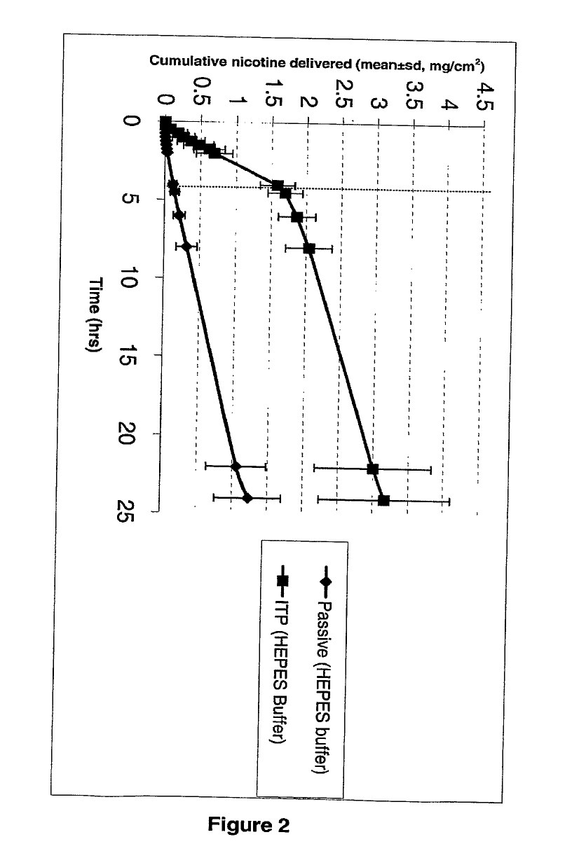

Nicotine is a diacidic base with pKa values of 3.4 and 8.2. It exists as a free base above pH 9. In between pH 4.8-7.5 it is present in the form of freebase and monocations. Passive transport of nicotine was higher at pH 8, when the drug predominantly exists in the non-ionized form, as compared to the monocationic form of nicotine at pH 5.5. Iontophoresis enhanced nicotine permeation compared to passive nicotine delivery in both of the conditions evaluated (FIGS. 1 and 2).

### Example 2

### Transdermal Delivery of Nicotine Salts by Passive Diffusion and Iontophoresis in 500 mM Citrate Donor Buffer

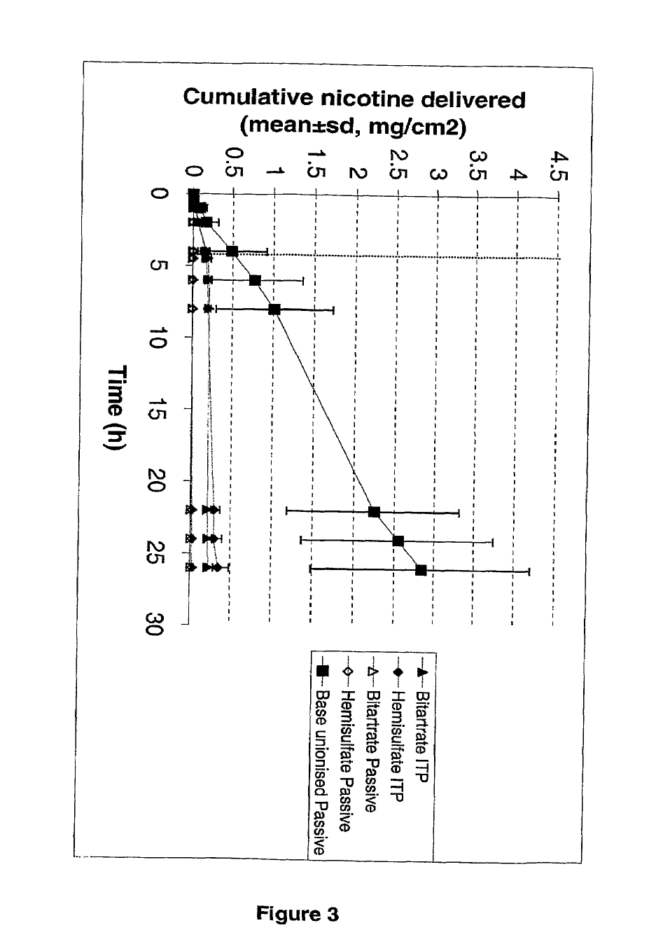

Passive and iontophoretic permeation of nicotine salts (equivalent to 1% nicotine), specifically nicotine bitartrate and nicotine hemisulfate, were studied in 500 mM citrate buffer with 50 mM NaCl. Iontophoretic current was terminated after 4 hours. The iontophoretic flux of nicotine was similar in both the salt forms, which was less than the flux from the passive delivery of nicotine base at pH 8 (FIG. 3). When pH of the donor solution was measure after the experiment, it was found that the pH of donor solutions did not change.

### Example 3

### Comparison of the Iontophoretic Delivery of Nicotine Bitartrate Salt in 50 mM HEPES Donor Buffer Vs 500 mM Citrate Donor Buffer

Iontophoresis of nicotine bitartrate salt was studied in 50 mM HEPES buffer pH 5.5. Nicotine bitartrate salt decreased the pH of HEPES buffer to about 3.5, which was then adjusted to about pH 5.5 with NaOH.

From the comparison plot (FIG. 3), it is seen that the flux of nicotine from 50 mM HEPES buffer is higher than the nicotine flux from a 500 mM citrate buffer. It appears that a higher buffer strength of citrate buffer contributed to more buffer ions which competed with the drug ions to be delivered across the skin, thereby reducing the nicotine permeation. There was some indication of this in the conductivity measurements. The 500 mM citrate buffer solution had a conductivity of 66400 μmhos/cm, compared to 16300 μmhos/cm measured for the 50 mM HEPES buffer solution. The pH measure at the end of the experiment with 50 mM HEPES solution indicated that it did not change significantly.

### Example 4

### Comparison of the Iontophoretic Delivery of Nicotinium Dihydrochloride Salt Versus Nicotine Bitartrate Salt

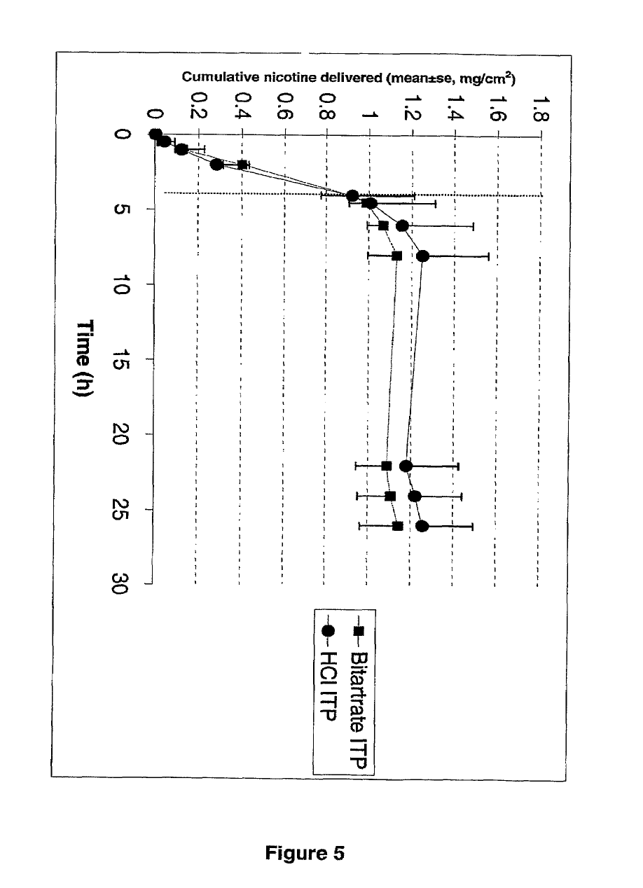

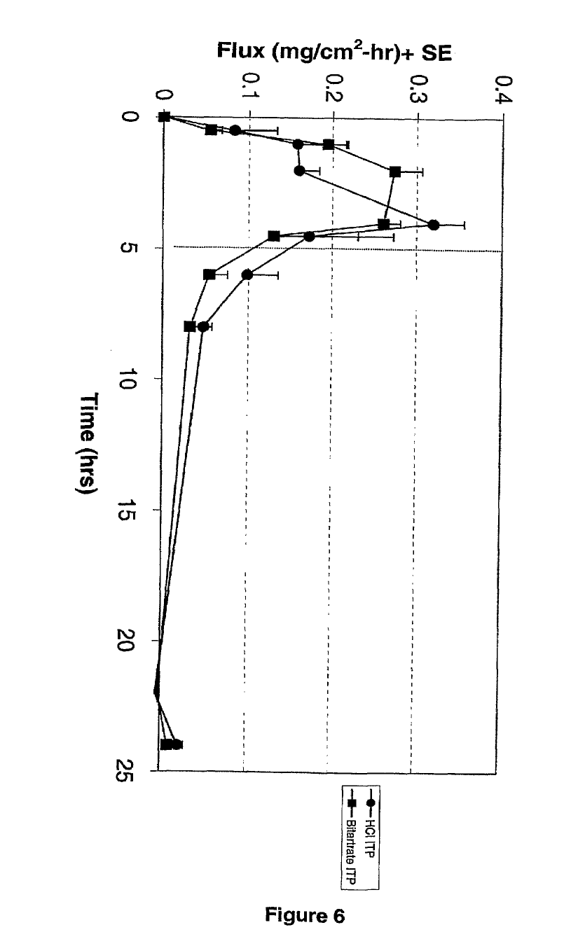

Iontophoretic delivery of nicotinium dihydrochloride salt was also studied in 50 mM HEPES buffer, adjusted to pH 5.5 with NaOH. The delivery profile was similar to that obtained from nicotine bitartrate salt in 50 mM HEPES buffer (FIG. 5). The conductivity of each donor solution was similar, 15400 μmhos/cm and 16300 μmhos/cm for nicotinium dihydrochloride in 50 mM HEPES and nicotine bitartrate in 50 mM HEPES, respectively. When the current was terminated at 4 hrs, the flux of nicotine from the both the salts decreased sharply (FIG. 6).

### Example 5

### Comparison of the Iontophoretic Delivery of Various Nicotine Salts in 50 mM HEPES Buffer

Iontophoretic permeation of various nicotine salts, i.e. maleate, dihydrobromide, dihydrosulfate, tetrahydrosulfate, citrate and dihydrohexanoate (equivalent to 1% nicotine, except citrate salt which was approximately 0.92%) were studied in 50 mM HEPES buffer, pH 5.5. Each respective nicotine salt was added to 50 mM HEPES, then pH was adjusted to 5.5 with NaOH. All the salts were directly dissolved in HEPES buffer, except nicotine dihydrohexanoate salt which was dissolved in HEPES buffer with the aid of 20% ethanol. The permeation profiles of the salts were compared to that of nicotine bitartrate and nicotine dihydrochloride salt (FIG. 6).

The conductivity, flux and lagtime of permeation of nicotine (passive, donor pH 8) and nicotine salts (iontophoresis, donor pH 5.5) are listed in Table 1. The flux is calculated at steady state when current is present for the iontophoresis experiments.

Iontophoresis of nicotine salts, in particular, nicotine maleate and nicotine citrate reduced the lagtime, 4 min and 9 min, respectively, compared to passive permeation of nicotine base (87 min). Iontophoresis of the nicotine salts also increased the flux of nicotine, flux ranging from 0.2073 mg/cm²-hr for nicotine bitartrate to 0.332 mg/cm²-hr for nicotine citrate, compared to passive permeation of nicotine base (0.1053 mg/cm²-hr).

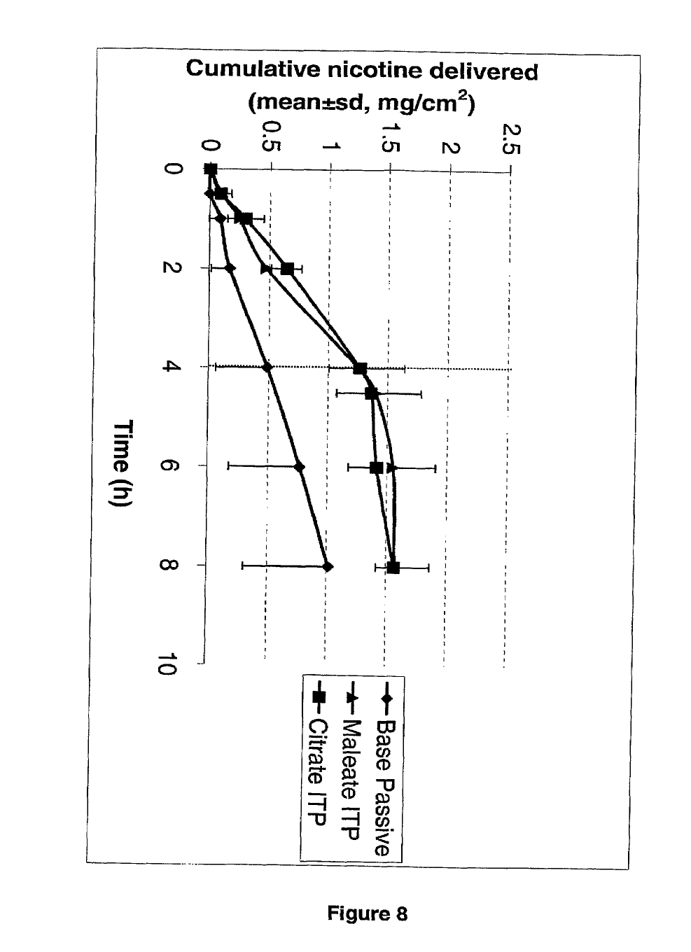

FIG. 8 compares passive permeation of nicotine base (pH 8) with iontophoresis of nicotine maleate and nicotine citrate at pH 5.5. Flux of nicotine is increased with iontophoresis of the nicotine salts, compared with passive permeation of nicotine base. Lagtime during iontophoresis of the nicotine salts is also reduced.

TABLE 1
Nicotine Salts used in Iontophoresis Experiments:

| | | Donor | Conductivity | Flux (mg/cm²- | Lagtime |
|---|---|---|---|---|---|
| | | pH | (mS/cm) | hr) | (min) |
| Passive | Base | 8 | 4.73 | 0.1053 | 87 |
| ITP | Bitartrate | 5.5 | 16.3 | 0.2073 | 10 |
| | Maleate | 5.5 | 9.31 | 0.2867 | 4 |
| | HCl | 5.5 | 15.4 | 0.2236 | 22 |
| | SO4 | 5.5 | 20 | 0.2554 | 46 |
| | HBr | 5.5 | 25.9 | 0.2771 | 63 |
| | Citrate | 5.5 | 13.3 | 0.332 | 9 |
| | Tetrahydro | 5.5 | 19.9 | 0.2178 | — |
| | SO4 | | | | |

What is claimed is:

1. A method of delivering nicotine to a patient wherein the nicotine is in the form of a nicotine salt and is administered via a transdermal patch comprising a system for providing an electrical current sufficient to enhance the flux of nicotine across the skin.

2. The method of claim 1 wherein the nicotine salt is selected from the group consisting of nicotine citrate, nicotine maleate or mixtures thereof.

3. The method of claim 2 wherein the nicotine salt is nicotine maleate.

4. A method of reducing irritation caused by a nicotine containing transdermal patch, wherein the patch comprises a nicotine salt.

5. The method of claim 4 wherein the nicotine salt is selected from the group consisting of nicotine maleate, nicotine citrate and mixtures thereof.

6. A transdermal patch suitable for administering nicotine to an individual wherein the patch comprises a nicotine salt and an electrical current sufficient to enhance the flux of nicotine across the skin.

7. The transdermal patch of claim 6 wherein the nicotine salt is selected from the group consisting of nicotine maleate, nicotine citrate or mixtures thereof.

8. The transdermal patch of claim 7 wherein the nicotine salt is nicotine maleate.

9. The transdermal patch of claim 6 wherein the nicotine salt is dissolved in a buffer solution resulting in a donor solution of a pH of from about 4 to about 9.

10. The transdermal patch of claim 6 wherein the pH of the donor solution is from about 5 to about 7.5.

11. The transdermal patch of claim 6 further comprising a microprocessor suitable for manipulating the amount of electrical current produced in order to modulate the rate of flux of nicotine across the skin.
